(19) United States
(12) Patent Application Publication
(10) Pub. No.: US 2020/0055800 A1
(43) Pub. Date: Feb. 20, 2020

(54) Linear Alpha Olefin Process Using Solvent Flash Drum for Olefin Separation

(71) Applicant: ExxonMobil Chemical Patents Inc., Baytown, TX (US)

(72) Inventors: Michael W. Weber, Houston, TX (US); James R. Lattner, La Porte, TX (US); Kirk C. Nadler, Houston, TX (US)

(21) Appl. No.: 16/609,950

(22) PCT Filed: Mar. 23, 2018
(86) PCT No.: PCT/US2018/023998

§ 371 Date: Oct. 31, 2019

Related U.S. Application Data
(60) Provisional application No. 62/503,757, filed on May 9, 2017.

(51) Int. Cl. C07C 2/32; C08F 2/01; C07C 11/04; C08F 10/02; C07C 7/00; C07C 7/04

(52) U.S. Cl. CPC C07C 2/32; C08F 2/01; C07C 11/04; C07C 2531/14; C07C 7/005; C07C 7/04; C08F 10/02

(57) ABSTRACT

The present disclosure provides assemblies for producing linear alpha olefins and methods for producing linear alpha olefins. In at least one embodiment, a method for producing a linear alpha olefin includes providing an olefin, a catalyst, and a process solvent to a reactor under oligomerization conditions; obtaining an effluent produced in the reactor; and transferring the effluent to a solvent-containing portion of a flash drum via a first effluent line coupled with the flash drum. In at least one embodiment, an assembly for producing linear alpha olefins includes a configuration to provide olefin, catalyst and process solvent coupled with a reactor; a flash drum; a first effluent line coupled with the reactor at a first end and coupled with the flash drum at a second end; and a second effluent line coupled with the flash drum at a first end and coupled with the first effluent line at a second end.

### PRIORITY CLAIM

This application claims the benefit of Provisional Application No. 62/503,757, filed May 9, 2017, the disclosures of which is incorporated herein by its reference.

### FIELD

The present disclosure provides assemblies for producing linear alpha olefins and methods for producing linear alpha olefins.

### BACKGROUND

Linear alpha olefins (LAOs) are commercially valuable for use as monomers in olefin polymerization processes, especially ethylene copolymerization. For example, linear alpha olefin monomers, such as 1-butene, 1-hexene, and 1-octene, can be copolymerized with ethylene to form a polyethylene copolymer backbone, for example, linear low density polyethylene (LLDPE). LLDPE produced using the linear alpha olefins 1-butene, 1-hexene and 1-octene accounts for a large percentage of the polyethylene resin market. In general, companies interested in polyethylene, purchase butene, hexene and octene for use in their polyethylene reactors. The butene, hexene, and octene are produced in separate reactors that typically produce a range of even-numbered alpha olefins from ethylene. It can be expensive to purchase these materials, and they add to the complexity of transport, storage and handling. An attractive alternative is to make these linear alpha olefins directly from the ethylene at the site where the ethylene is formed and will be used for subsequent polymerization, if this can be done cleanly and economically.

Nonetheless, conventional assemblies configured to form linear alpha olefins can experience polymeric fouling of byproducts (such as polyethylene) formed during a linear alpha olefin forming process, which causes a need for assembly shutdown to clean the fouled components of the assembly. Furthermore, linear alpha olefin producing assemblies are energy intensive.

There is a need for improved assemblies for producing linear alpha olefins and methods for producing linear alpha olefins to more effectively generate linear alpha olefins. More particularly, a need exists for controlling and/or mitigating polymeric fouling in linear alpha olefin assemblies. Such fouling reduction would provide benefits including but not limited to reducing/eliminating process down time, more efficiently and/or cost effectively producing desired linear alpha olefins, reducing oligomerization reaction byproducts (e.g., branched alpha olefins), and/or reducing/minimizing inefficiencies in energy consumption/throughput of an assembly.

### SUMMARY

The present disclosure provides assemblies for producing linear alpha olefins and methods for producing linear alpha olefins.

In at least one embodiment, a method for producing a linear alpha olefin includes providing an olefin, a catalyst, and a process solvent to a reactor, preferably a tubular reactor, under oligomerization conditions; obtaining an effluent produced in the reactor; and transferring the effluent to a solvent-containing portion of a flash drum via a first effluent line coupled with the flash drum.

In at least one embodiment, an assembly for producing linear alpha olefins, the assembly includes a configuration to provide olefin, catalyst and process solvent coupled with a reactor, preferably a tubular reactor; a flash drum; a first effluent line coupled with the reactor at a first end and coupled with the flash drum at a second end; and a second effluent line coupled with the flash drum at a first end and coupled with the first effluent line at a second end.

### BRIEF DESCRIPTION OF THE FIGURES

FIG. 1A is an assembly for producing linear alpha olefins comprising a reaction zone, according to an embodiment of the present disclosure.

FIG. 1B is an assembly for producing linear alpha olefins comprising a distillation zone, according to an embodiment of the present disclosure.

FIG. 2 is a reaction zone of an assembly for producing linear alpha olefins, according to an embodiment of the present disclosure.

FIG. 3 is a quench zone of an assembly for producing linear alpha olefins, according to an embodiment of the present disclosure.

FIG. 4 is a quench zone of an assembly for producing linear alpha olefins, according to an embodiment of the present disclosure.

FIG. 5 is a distillation zone of an assembly for producing linear alpha olefins, according to an embodiment of the present disclosure.

FIG. 6 is a distillation scheme using paraxylene, metaxylene, or orthoxylene as solvent, according to an embodiment of the present disclosure.

### DETAILED DESCRIPTION

The present disclosure provides assemblies for producing linear alpha olefins and methods for producing linear alpha olefins. In at least one embodiment, a method for producing a linear alpha olefin includes providing an olefin, a catalyst, and a process solvent to a reactor, preferably a tubular reactor, under oligomerization conditions; obtaining an effluent produced in the tubular reactor; and transferring the effluent to a solvent-containing portion of a flash drum via a first effluent line coupled with the flash drum. In at least one embodiment, an assembly for producing linear alpha olefins includes a configuration to provide olefin, catalyst and process solvent coupled with a reactor, preferably a tubular reactor; a flash drum; a first effluent line coupled with the reactor at a first end and coupled with the flash drum at a second end; and a second effluent line coupled with the flash drum at a first end and coupled with the first effluent line at a second end. Assemblies and methods for producing linear alpha olefins of the present disclosure reduce or prevent the presence of solid components (such as fouling to polymer, branched alpha olefins, and C₃₀₊ waxes) from occupying a top portion of the flash drum, reducing the likelihood that solid components enter adjacent lines and structures, for example, a knockout drum.

As used herein, the term “polyolefin copolymer” includes homopolymers and copolymers wherein at least 80% by weight (wt %), preferably at least 85 wt %, more preferably at least 90 wt %, for example at least 95 wt %, at least 98 wt %, at least 99 wt %, at least 99.5 wt %, at least 99.9 wt %, or 100 wt %, as synthesized, of the monomer repeat units are based on a repeat unit structure of a specific alpha-olefin. For example, where the olefin is ethylene, the repeat unit structure would be —(CH₂—CH₂)—. In embodiments where one or more comonomers are included in a linear alpha olefin formed in an assembly of the present disclosure, the one or more comonomers can be collectively present in the linear alpha olefin product in an amount of not more than 20 wt %, preferably not more than 15 wt %, more preferably not more than 10 wt %, for example not more than 5 wt %, not more than 2 wt %, not more than 1 wt %, not more than 0.5 wt %, or not more than 0.1 wt %. The one or more comonomers, when present, can include, but are not limited to, C₄-C₁₀ alpha-olefins (e.g., 1-butene, 1-hexene, 1-octene, and 1-decene), such as C₄-C₈ alpha-olefins such as 1-butene, 1-hexene, and/or 1-octene. In one embodiment, the one or more comonomers, when present, can be substantially free from dienes and polyunsaturated compounds.

As referred to herein, selective oligomerization refers to producing the desired linear alpha olefins (“the oligomers”) with a selectivity of the reaction being at least 80%, more specifically at least 90%, by mole of desired oligomer(s), with the possibility that an acceptable amount of polymer is present, but with the preference that no polymer is present in the product. In other embodiments, less than 10 wt % of polymer is formed by the selective oligomerization reaction, specifically less than 5 wt %, more specifically less than 2 wt %, based upon the total weight of monomer converted to oligomers and polymers, where a polymer is defined to mean a molecule comprising more than 50 monomer repeat units. An “oligomer” as used herein is defined to mean a molecule comprising from 2 to 50 monomer repeat units, but preferably 30 total carbons or less, such as 20 total carbons or less, such as C₄-C₂₀ linear alpha olefins. In other embodiments, selective oligomerization refers to producing one or two desired oligomers, with the selectivity of the one or two desired oligomers summing to at least 80%, e.g., at least 90%, by sum of total moles of oligomers. Particularly preferred desired oligomers are molecules consisting of 2 to 10 monomeric repeat units of ethylene and having 4 to 20 total carbon atoms, with an olefinic unsaturation at the end of the oligomer (i.e., alpha-olefin oligomers).

As referred to herein, the terms “fouling polymer” and “fouled polymer” are synonymous and refer to polymer that not only has become insoluble in the oligomerization reaction medium under oligomerization conditions but also has deposited on one or more surfaces within the oligomerization reactor, which includes not only the walls of a tubular reactor but also on surfaces of other components inside the assembly such as a flash drum or piping, such that the fouling/fouled polymer remains within the assembly (i.e., does not exit the reactor during the ordinary course of the reaction).

Methods according to the present disclosure can comprise separating the desired oligomerization product from the effluent of a tubular reactor to attain an olefinic purity of desired oligomerization product of at least 90 mol %, for example at least 93 mol %, at least 95 mol %, at least 96 mol %, at least 97 mol %, or at least 98 mol % in the separated effluent after a distillation.

While the feed comprising the alpha-olefin into a tubular reactor can contain one or more C₂-C₁₂ alpha-olefins, the most preferred alpha-olefin for the oligomerization reactions described herein is ethylene. As a result, in a preferred embodiment, the alpha-olefin feed comprises greater than 99 wt % ethylene.

### Assemblies for Producing Linear Alpha Olefins

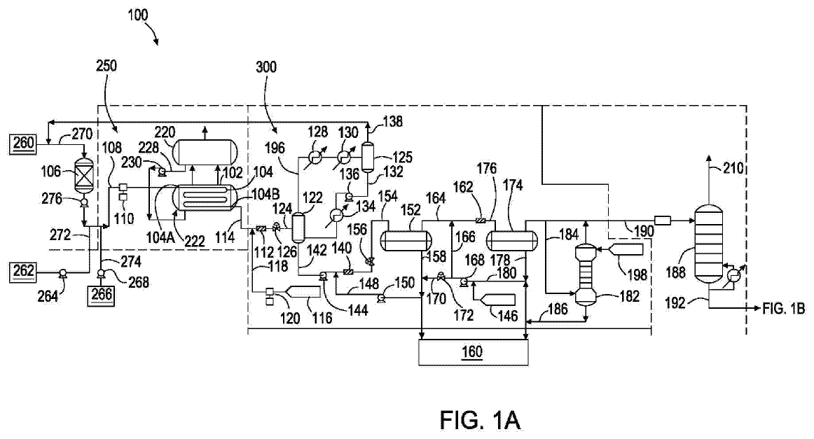

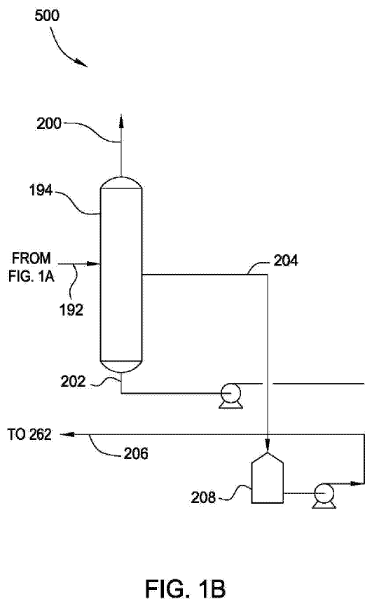

FIG. 1A is an assembly for producing linear alpha olefins comprising a reaction zone, according to an embodiment of the present disclosure. As shown in FIG. 1A, an assembly 100 comprises a reaction zone 250. Reaction zone 250 comprises reactor section 222 comprising reactor 104 configured to form linear alpha olefins from ethylene upon introduction of ethylene and catalyst into reactor 104. Preferably, reactor 104 is a tubular reactor. A tubular reactor can act as a plug flow reactor which reduces oligomer chain branching during use, as compared to a CSTR (continuously stirred tank reactor), and the reaction has a high concentration of olefins to re-incorporate toward the end of the tube. In a CSTR, the concentration of olefins is substantially uniform throughout the entire reactor. Reactor 104 is coupled with steam jacket 102 to control the temperature profile along reactor 104. Reactor 104 provides minimized branching of olefins formed in the reactor due to reduced backmixing as compared to conventional linear alpha olefin reactors. In order to minimize dispersion in the tubular reactor, the tubular reactor diameter can be set to achieve a Reynolds number in the turbulent flow regime, e.g., greater than 4,000. In one embodiment, a tubular reactor has a diameter from 1 inch to 3 inches. The diameter can be greater than or less than this range depending on a desired pressure drop, fluid flow velocity, and/or residence time. The tubular reactor can have enough parallel sections to provide a pressure drop through the reactor that does not cause a phase change of the unreacted ethylene portion of the reactor contents.

Steam drum 220 contains fluid (such as a cooling fluid, such as water) which is provided to steam jacket 102 of reactor section 222 via fluid line 228. The amount and flow rate of fluid provided into steam jacket 102 is controlled by fluid pump 230. Fluid pump 230 provides a pressure to the fluid to provide flow of the fluid to steam jacket 102 which regulates the temperature within reactor section 222.

During a linear alpha olefin forming process, reactor 104 can have a change in temperature (ΔT) from a first end 104A to a second end 104B of the tubular reactor of from 5° C. to 35° C., such as 20° C., and a peak temperature of from 150° C. to 190° C., such as 170° C. at the first end of the reactor. Olefin source 260 is a container that provides olefin monomers, such as ethylene, to a guard drier 106 via olefin line 270. The amount and flow rate of olefin into guard drier 106 is controlled by pump 276. Pump 276 can be a diaphragm pump.

From guard drier 106, olefin is transferred to reactor 104 via transfer line 108. Process solvent source 262 is a container that provides process solvent to transfer line 108 via process solvent line 272. The amount and flow rate of process solvent into transfer line 108 is controlled by pump 264. Catalyst source 266 is a container that provides one or more catalysts to transfer line 108 via catalyst source line 274. The amount and flow rate of catalyst into transfer line 108 is controlled by pump 268. Olefin, process solvent, and catalyst form a ‘reactor feed’ (also known as an ‘olefin mixture’) upon mixing in transfer line 108.

A process solvent can be one or more of paraxylene, orthoxylene, metaxylene, and the like. Suitable catalysts can include zirconium and chromium based catalysts. Any water present in the reactor feed in transfer line 108 that is subsequently flowed into a reactor can promote formation of a fouling polymer, for example, polyethylene. Therefore, during use, guard drier 106 can contain one or more drying agents, such as 3 Å or 4 Å mole sieves, to remove water from the olefin.

The amount and flow rate of olefin (with catalyst and process solvent) into reactor 104 is controlled by pump 110. Pump 110 can be a membrane pump. Pump 110 provides pressure to the olefin (with catalyst and process solvent) flowing into reactor 104 sufficient to keep the olefin (with catalyst and process solvent) in a liquid phase to reduce or prevent the formation of precipitates (such as fouling polymer) within reactor 104. In at least one embodiment, the pressure provided by membrane pump 110 to the olefin (with catalyst and process solvent) flowing into reactor 104 is 3,000 pounds per square inch (PSI) or greater. At a pressure of 3,000 PSI or greater, olefin, such as ethylene, is in a supercritical phase and other components such as catalyst and process solvent are homogeneous within this olefin mixture. Flow of the olefin mixture through reactor 104 can be a turbulent flow (as opposed to a laminar flow) so there is no dispersion (and a Reynolds Number (Re) of 2,000 or greater). The olefin (with catalyst and process solvent) can have a residence time in reactor 104 of from 5 minutes to 15 minutes, such as 10 minutes. Furthermore, because linearity of an alpha olefin formed in a reactor decreases as the carbon number of the formed alpha olefins increases (e.g., C₃₀ alpha olefins) and with increasing conversion, conversion of olefin to linear alpha olefin per pass through a reactor can be from 50% to 80%, such as from 55% to 70%, such as from 60% to 65%.

After reaction of olefin into linear alpha olefins within reactor 104, an effluent is transferred from reactor 104 via effluent line 114 and combined with quench agent via line 118 and then flowed to a mixer 112, a valve 126, and/or flash drum 122. Effluent transferred from reactor 104 often has a temperature from 160° C. to 170° C. and experiences a pressure letdown (i.e., the pressure within the effluent line reduces) across letdown valve 126. This pressure letdown promotes branched alpha olefin formation within the effluent. Furthermore, the effluent is a homogeneous liquid and the pressure letdown promotes precipitate formation in effluent line 114, which can lead to fouling. It has been discovered that quenching the effluent with a quench agent (before pressure letdown valve 126 and prior to entering flash drum 122) deactivates catalyst within the effluent and reduces or eliminates the formation of branched alpha olefins and/or precipitate formation in effluent line 114 and/or effluent line 124. A quench agent source 116 provides a quench agent to effluent line 114 via quench agent line 118 coupled with effluent line 114, where the effluent within effluent line 114 is combined with quench agent that flows into mixer 112. Alternatively, the effluent within effluent line 114 is preferably combined with quench agent that flows to flash drum 122 via line 124 (without entering a mixer coupled with effluent line 114). The amount and flow rate of quench agent provided to effluent line 114 is controlled by pump 120. Pump 120 provides quench agent to effluent line 114 at a pressure sufficient to compensate for the pressure letdown of effluent from reactor 104 flowing through effluent line 114, which reduces or eliminates the formation of branched olefins and/or precipitate formation in effluent line 114.

Suitable quench agents include organic quench agents such as amines, such as 1,5-diamino-2-methylpentane (also known as 2-methyl-1,5-pentamethylenediamine) Organic quench agents are advantageous over, for example, conventional aqueous sodium hydroxide quench agent formulations because aqueous solutions lead to large quantities (e.g., greater than 100 ppb) of water in recycled ethylene to be fed back to reactor 104 from a conventional recycle loop line. Also, organic quench agents have higher boiling points than water and do not flash overhead with the recycled olefin. Nonetheless, the amines, for example, can be water soluble and so can be removed in a downstream aqueous wash separate from an ethylene recycle loop line.

After sufficient mixing, an effluent is transferred to flash drum 122 via an effluent line 124. The amount and flow rate of effluent provided to flash drum 122 is controlled by letdown valve 126. Valve 126 can be any suitable valve, such as a V-ball valve, which can be obtained commercially such as the Fisher™ V-Series from Fisher Valves & Instruments. During a linear alpha olefin forming process, a temperature within effluent line 114, mixer 112, quench agent line 118, flash drum 122, and effluent line 124 can be maintained at a temperature of 130° C. or greater to prevent C₃₀₊ waxes and polyethylene from crystallizing out of the process solvent solution. Flash drum 122 will contain process solvent, unreacted olefin, linear alpha olefins, quenching agent, and any byproducts/impurities (if present) such as polyethylene, branched alpha olefins, linear internal olefins, and C₃₀₊ waxes. At a temperature of 130° C. or greater, olefins such as ethylene and some quenching agent can be volatilized to a top portion of flash drum 122 and can be provided, as an effluent, to knockout drum 125 via effluent line 196. Knockout drum 125 can be a quench agent knockout drum. Effluent line 196 is coupled with chillers 128 and 130 configured to reduce the temperature of the effluent flowing through effluent line 196 and entering knockout drum 125. The chilled effluent flowing through effluent line 196 promotes precipitation of quench agent and olefin products as it enters knock out drum 125 to simplify olefin purification within knock out drum 125. During use, knockout drum 125 can contain a de-mister to prevent entrainment of liquid particles into the knockout drum overhead stream. The temperature of knockout drum 125 can be high enough to volatilize unreacted ethylene while low enough to reduce or prevent volatilization of other components present in knockout drum 125, such as residual quench agent. Recycle line 138 is coupled with a top portion of knockout drum 125 and is configured to return unreacted olefin to olefin line 270 for subsequent drying in guard drier 106 followed by reaction to form linear alpha olefins within reactor 104, as described above. A high boiling fraction from knockout drum 125 can be provided, as an effluent, to flash drum 122 via effluent line 132. Effluent line 132 is coupled with heater 134 configured to increase the temperature of the effluent flowing through effluent line 132 and entering flash drum 122. Effluent line 132 is further coupled with pump 136 configured to provide pressure to effluent within effluent line 132 and regulate a flow rate of effluent through effluent line 132 and into flash drum 122.

A high boiling fraction (e.g., heavy fraction) from flash drum 122 is provided, as an effluent, to a caustic solution mixer 140 via effluent line 142. A high boiling fraction can contain process solvent, linear alpha olefin products, catalyst, and any byproducts/impurities (if present) such as polyolefins (e.g., polyethylene), branched alpha olefins, linear internal olefins, and C₃₀₊ waxes. The amount and flow rate of the effluent provided to mixer 140 is controlled by pump 144. A caustic solution source 146 provides an aqueous caustic solution (e.g., sodium hydroxide) to effluent line 142 via caustic solution line 148 coupled with effluent line 142, where the effluent within effluent line 142 combines with caustic solution that flows into mixer 140. The amount and flow rate of caustic solution provided to effluent line 142 is controlled by pump 150 which is coupled with caustic solution line 148.

After sufficient mixing, the combination of effluent and caustic solution is provided, as an effluent, to settling drum 152 via effluent line 154. The amount and flow rate of the effluent provided to settling drum 152 is controlled by mixing valve 156 that is coupled with effluent line 154. During use, settling drum 152 separates catalyst, such as zirconium, chromium, and/or aluminum metals, from other components of the high boiling fraction, such as linear alpha olefin product. In the settling drum, the hydrocarbon and aqueous phases are allowed to separate by density where a biphasic mixture forms having an organic top layer (containing linear alpha olefin product) and an aqueous bottom layer (containing caustic solution, quenching agent, catalyst, and other impurities soluble in aqueous solution). The bottom aqueous layer is provided, as an effluent, via effluent line 158 to either (a) waste water treatment facility 160 or (b) caustic solution line 148 for reuse as a caustic solution provided to effluent line 142. If the bottom aqueous layer is provided to effluent line 142, additional caustic solution can be provided to effluent line 158 from caustic solution source 146 via caustic solution line 170, which dilutes the effluent (containing bottom aqueous layer from settling drum 152) with caustic solution. The amount and flow rate of caustic solution provided to effluent line 158 is controlled by valve 172 that is coupled with caustic solution line 170.

The top organic layer present in settling drum 152 is provided, as an effluent, to mixer 162 via effluent line 164. Caustic solution source 146 provides an aqueous caustic solution (e.g., sodium hydroxide) to effluent line 164 via caustic solution line 166 coupled with effluent line 164, where the effluent within effluent line 164 combines with caustic solution and flows into mixer 162. The amount and flow rate of caustic solution provided to effluent line 164 is controlled by pump 168 that is coupled with caustic solution lines 166 and 170. After sufficient mixing, the mixture of effluent and caustic solution is provided, as an effluent, to second settling drum 174 via effluent line 176. During use, settling drum 174 separates metal hydroxides formed from the reaction of caustic with residual catalyst, such as zirconium, chromium, and/or aluminum metals, from other components of the organic phase, such as linear alpha olefin product. The settling drum allows the hydrocarbon and aqueous phases to separate, where a biphasic mixture forms having an organic top layer (containing linear alpha olefin product) and an aqueous bottom layer (containing caustic solution and residual quenching agent, catalyst, and other impurities soluble in aqueous solution). The bottom aqueous layer is provided, as an effluent, via effluent line 178 to either (a) waste water treatment facility 160 or (b) caustic solution line 180 for reuse as a caustic solution provided to effluent lines 164 or 158. If the bottom aqueous layer is provided to effluent lines 164 or 158, additional caustic solution can be provided to effluent lines 164 or 158 from caustic solution source 146, which dilutes the effluent in effluent line 164 or 158 with caustic solution.

The top organic layer present in settling drum 174 is provided, as an effluent, to wash tower 182, which can be a wash water tower, via effluent line 184. Wash source 198, which can be a wash water source, provides a fluid, such as water, to wash tower 182 where, water and the oil phase are continuously contacted in a counter-current configuration through several equilibrium stages to form an organic overhead stream (containing linear alpha olefin product) and an aqueous bottom layer (a bottoms stream containing fluid and any residual quenching agent, catalyst, and caustic solution). The bottom aqueous stream is provided, as an effluent, via effluent line 186 to waste water treatment facility 160. The top organic stream is provided, as an effluent, to deethanizer tower 188 via effluent line 190. In deethanizer tower 188, unreacted ethylene is separated from olefin products and the process solvent. The ethylene stream 210 is subjected to drying with mol sieves before being recycled to line 270. The olefin product and process solvent stream 192 is fed to the distillation section.

FIG. 1B is an assembly for producing linear alpha olefins comprising a distillation zone, according to an embodiment of the present disclosure. From deethanizer tower 188, the linear alpha olefin products (with remaining process solvent) are transferred, as an effluent, via effluent line 192, to fractional distillation tower 194 of FIG. 1B. Distillation tower 194 has one or more reboilers (not shown) disposed beneath it. Reboilers are heat exchangers typically used to provide heat to the bottom of industrial distillation columns. During use, fractional distillation tower 194 separates light linear alpha olefins (C₄, C₆, C₈) from heavier linear alpha olefins (C₁₀-C₂₀). The light linear alpha olefins can be removed, as an effluent, from fractional distillation tower 194 via effluent line 200. This light linear alpha olefin fraction can be collected and stored or can undergo further purification in one or more additional distillation tower(s) (not shown). One or more of the additional distillation tower(s) can have a dividing wall. The heavier linear alpha olefins (C₁₀-C₂₀) can be removed, as an effluent, from fractional distillation tower 194 via effluent line 202. This heavier linear alpha olefin fraction can be collected and stored or can undergo further purification in one or more additional distillation tower(s) (not shown). One or more of the additional distillation tower(s) can have a dividing wall. One or more additional distillation tower(s) having a dividing wall provides capital cost savings by using fewer overall distillation towers and operating costs by consuming less overall energy. One or more additional distillation tower(s) having a dividing wall can further provide processes using a xylene solvent or any other solvent which boils between 1-octene and 1-decene. LAO processes using higher-boiling or lower-boiling solvents can also use the one or more additional distillation tower(s) having a dividing wall. For example, some LAO processes use solvents (for example, cyclohexane or toluene) that boil between 1-hexene and 1-octene. An analogous process configuration using any solvent can use the one or more additional distillation tower(s) having a dividing wall to recover high purity LAO products and high purity solvent, and the solvent preferably does not co-boil with an LAO. The recovered high purity solvent can be recycled to the front end of the process, such as process solvent source 262, for re-use.

Process solvent present in distillation tower 194 of FIG. 1B can be (as a middle distillate) removed, as an effluent, from fractional distillation tower 194 via effluent line 204. The process solvent can be collected and stored in process storage tank 208 or can be recycled via recycle loop line 206 back to process solvent source 262. Alternatively, if the process solvent is for example paraxylene, which boils between C₈ and C₁₀ linear alpha olefin, the middle distillate contains a minor fraction of C₈ and C₁₀ linear alpha olefins. Therefore, the middle distillate can be removed, as an effluent, from fractional distillation tower 194 via effluent line 204 and be provided to one or more additional distillation tower(s) (not shown) before being recycled via a recycle loop line back to process solvent source 262.

### Reaction Zones

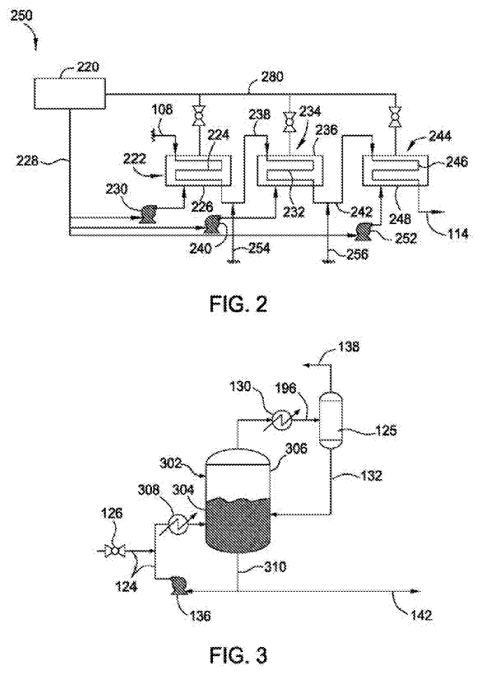

FIG. 2 is a reaction zone of an assembly for producing linear alpha olefins, according to another embodiment of the present disclosure. Reaction zone 250 comprises steam drum 220 and reactor section 222 comprising tubular reactor 224 and steam jacket 226. Steam drum 220 provides fluid (such as a cooling fluid, such as water) to steam jacket 226 of reactor section 222 via fluid line 228. Steam formed within steam jacket 226 can be recycled back to the steam drum via steam recycle loop line 280. The amount and flow rate of fluid provided into steam jacket 226 is controlled by fluid pump 230, which may be a cooling fluid pump. Fluid pump 230 provides a flow of fluid, such as cooling fluid, to steam jacket 226 which regulates a temperature (T1) within steam jacket 226 of reactor section 222. A valve coupled with an outlet line of steam jacket 226 regulates pressure of steam/fluid within steam jacket 226. A mixture of olefin, catalyst, and process solvent is provided to tubular reactor 224 from a reactor feed of transfer line 108. The olefin (with catalyst and process solvent) can have a residence time in tubular reactor 224 of from 1 minute to 15 minutes, such as 3 minutes. After reaction of olefin to form linear alpha olefins within tubular reactor 224, an effluent (comprising linear alpha olefins, unreacted olefin, catalyst, solvent, and any byproducts (if present)) is transferred from tubular reactor 224 via effluent line 238 to a second reactor section 234 comprising a second reactor 232 and a second steam jacket 236. Second reactor 232 can be a tubular reactor. Steam drum 220 provides fluid to steam jacket 236 of second reactor section 234 via fluid line 228. The amount and flow rate of fluid provided into second steam jacket 236 is controlled by fluid pump 240. Fluid pump 240 provides a flow of fluid to second steam jacket 236 which regulates a temperature (T2) within steam jacket 236 of reactor section 234. A valve coupled with an outlet line of steam jacket 236 regulates pressure of steam/fluid within steam jacket 236. The mixture of linear alpha olefins, olefin, catalyst, solvent, and any byproducts (if present) can have a residence time in tubular reactor 232 of from 1 minute to 15 minutes, such as 3 minutes. After further reaction of olefin to form additional linear alpha olefins within tubular reactor 232, an effluent (comprising linear alpha olefins, olefin, catalyst, solvent, and any byproducts (if present)) is transferred from tubular reactor 232 via effluent line 242 to a third reactor section 244 comprising a third reactor 246 and a third steam jacket 248. Third reactor 246 can be a tubular reactor. Steam drum 220 provides fluid (such as cooling fluid, such as water) to steam jacket 248 of third reactor section 244 via fluid line 228. The amount and flow rate of fluid provided into third steam jacket 248 is controlled by fluid pump 252. Fluid pump 252 provides a flow of fluid to third steam jacket 248 which regulates a temperature (T3) within steam jacket 248 of reactor section 244. A valve coupled with an outlet line of steam jacket 248 regulates pressure of steam/fluid within steam jacket 248. The mixture of linear alpha olefins, olefin, catalyst, solvent, and any byproducts (if present) can have a residence time in tubular reactor 246 of from 1 minute to 15 minutes, such as 3 minutes. Controlling the temperature in each steam jacket (steam jackets 226, 236, and 248) provides greater linearity of linear alpha olefins because the temperature can be regulated with respect to catalyst age in each reactor section (reactor sections 222, 234, and 244). In general, catalyst in downstream reactor sections will be older than catalyst up upstream reactor sections. As used herein, ‘catalyst age’ includes the time catalysts and co-catalysts have been under reaction conditions. In at least one embodiment, T1 is greater than T2, and T1 and T2 are greater than T3. In at least one embodiment, T1 is 170° C., T2 is 165° C., and T3 is 160° C.

In one embodiment, the length along the outer surface of each of tubular reactors 224, 236, and 246 is shorter than the length along the outer surface of reactor 104. In one embodiment, the length along the outer surface of each of tubular reactors 224, 236, and 246 is ⅓ the length along the outer surface of reactor 104.

After further reaction of olefin to form additional linear alpha olefins within tubular reactor 246, an effluent (comprising linear alpha olefins, ethylene, catalyst, solvent, and any byproducts (if present)) is provided to a quench section via effluent line 114, as described in FIG. 1A.

Multiple tubular reactors (and multiple steam jackets) provides control of the degree of olefin conversion in each reactor section, which provides a higher degree of linearity of linear alpha olefin products formed while maintaining overall conversion, which occurs because linearity decreases with high temperatures and longer residence time. With multiple tubular reactors (and multiple steam jackets) each reactor temperature is controlled (typically reduced), such as in a downstream reactor, such as tubular reactor 146, where the conversion of olefin is typically higher.

In one embodiment, one or more of the effluent lines, such as effluent lines 238 and/or 242, is coupled with one or more additional solvent or ethylene feed lines (254, 256). An additional solvent feed line coupled with effluent lines 238 and/or 242 provides control of the solvent to olefin ratio introduced into each of reactors 232 and 246. Control of the solvent to olefin ratio introduced into each of tubular reactors 224, 232, and 246 provides increased degree of linearity of overall linear alpha olefins formed because linearity typically decreases as the solvent to olefin ratio decreases (and this effect is more pronounced at higher conversion). Linearity of the linear alpha olefins can therefore be increased at constant total conversion by feeding additional solvent to the downstream reactor sections in order to increase the solvent to olefin, e.g., ethylene ratio in the sections with higher conversion. Additional olefin, e.g., ethylene decreases the concentration of products at higher conversions and decreases the rate of formation of branched product. Later stage injection of additional olefin, e.g., ethylene provides additional benefit to linearity by providing more driving force for the addition of a C₂ over higher LAO into the chain. Like solvent addition, olefin, e.g., ethylene addition also dilutes the product concentration to reduce the kinetic rate of re-insertion of the LAO product into the chain which causes branches.

### Quench Zones

FIG. 3 is a quench zone of an assembly for producing linear alpha olefins, according to another embodiment of the present disclosure. As shown in FIG. 3, quench zone 300 comprises flash drum 302. An effluent is transferred from mixer 112 to flash drum 302 via effluent line 124. Effluent line 124 is coupled with a bottom portion 304 of flash drum 302. The amount and flow rate of effluent provided to flash drum 302 is controlled by valve 126, such as a V-Ball valve. During a linear alpha olefin forming process, a temperature within effluent line 114, mixer 112, quench agent line 118, flash drum 302, and effluent line 124 can be maintained at a temperature of 130° C. or greater to prevent C₃₀₊ waxes and polyethylene from crystallizing out of the process solvent solution. For example, heater 308 is coupled with effluent line 124 to maintain an effluent temperature at 130° C. or greater before the effluent is introduced into flash drum 302. Alternatively, heater 308 is preferably located upstream of the effluent introduction (e.g., coupled with effluent line 114) such that effluent coming from the letdown valve (e.g., valve 126) is mixed with the effluent flowing through effluent line 114 that has already been heated by heater 308 which then flows into the flash vessel. During use, flash drum 302 contains process solvent, olefin, linear alpha olefins, quenching agent, and any byproducts/impurities (if present) such as fouling polymer (e.g., polyethylene), branched alpha olefins, linear internal olefins, and C₃₀₊ waxes. Flash drum 302 can contain a sufficient amount of process solvent such that effluent introduction into flash drum 302 via effluent line 124 at bottom portion 304 occurs below the liquid level within flash drum 302, which mitigates the presence of solid components (such as fouling polymer and C₃₀₊ waxes) from occupying a top portion 306 of flash drum 302, reducing the likelihood that solid components enter knockout drum 125 via effluent line 196.

At a temperature of 130° C. or greater, olefin (such as ethylene) and some quenching agent can be volatilized to top portion 306 of flash drum 302 and can be provided, as an effluent, to knockout drum 125 via an effluent line, such as effluent line 196 of FIG. 1A. Effluent line 196 is coupled with one or more chillers (such as chiller 130) configured to reduce the temperature of the effluent flowing through effluent line 196 and entering knockout drum 125. Chiller 130 reduces the temperature of stream 196 enough to induce precipitation of the quench agent. During use, knockout drum 125 can contain a demister to prevent carryover of liquid droplets into the knockout drum overhead. The temperature of knockout drum 125 can be high enough to volatilize unreacted olefin while low enough to reduce or prevent volatilization of other components present in knockout drum 125, such as excess C₄+ olefin products and residual quench agent. Recycle line 138 is coupled with a top portion of knockout drum 125 and returns unreacted olefin to olefin line 270, through guard drier 106, followed by subsequent reaction to form linear alpha olefins within reactor 104. A high boiling fraction from knockout drum 125 can be provided, as an effluent, to flash drum 302 via effluent line 132. A separate stream within effluent line 310 (from the bottom of flash drum 302) is flowed through an effluent line coupled with heater 308 and then mixed with effluent from the upstream reactor in the stream of effluent line 124. The mixed reactor effluent and flash drum bottoms are fed into the flash drum. Mixing the reactor effluent flowing from valve 126 with the recycled flash bottoms 310 provides dilution of any solids in the reactor effluent and prevent carryover of solid material into the flash drum headspace.

A high boiling fraction from flash drum 302 is provided, as an effluent, to a caustic solution mixer (such as caustic mixer 140) via effluent line 142. A high boiling fraction can contain process solvent, linear alpha olefin products, catalyst, and any byproducts/impurities (if present) such as fouling polymer, branched alpha olefins, linear internal olefins, and C₃₀₊ waxes.

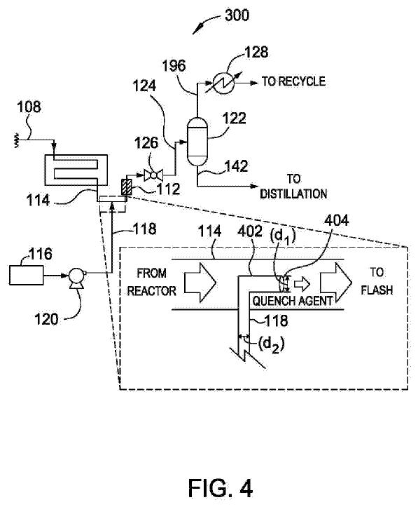

FIG. 4 is a quench zone of an assembly for producing linear alpha olefins, according to another embodiment of the present disclosure. Quench zone 300 comprises mixer 112 and effluent line 114. After reaction of olefin in a tubular reactor, such as reactor 104, an effluent is transferred from the tubular reactor through effluent line 114. Effluent transferred from the tubular reactor often has a temperature from 160° C. to 170° C. and experiences a pressure letdown upon exiting the tubular reactor. This pressure letdown promotes branched alpha olefin formation within the effluent. Furthermore, the effluent is a homogeneous liquid and the pressure letdown promotes precipitate formation in effluent line 114, which could lead to fouling. Quenching the effluent with a quenching agent (before the pressure reduction through valve 126 and entering flash drum 122) reduces or eliminates the formation of branched alpha olefins and/or precipitate formation in effluent line 114. A quench agent source 116 provides a quench agent to effluent line 114 via quench agent line 118 coupled with effluent line 114, where the effluent within effluent line 114 combines with quench agent and flows into mixer 112 or flash drum 122 (without use of mixer 112). The amount and flow rate of quench agent provided to effluent line 114 is controlled by pump 120. Pump 120 provides quench agent to effluent line 114 at a pressure sufficient to compensate for the pressure letdown of effluent from reactor 104 flowing through effluent line 114, which reduces or eliminates the formation of branched alpha olefins and/or precipitate formation in effluent line 114. Effluent line 118 is connected to a dip tube section 402 having a transverse outlet 404 for flow of quench agent into effluent line 114. The dip tube section can be disposed along a center, longitudinal axis of effluent line 114, which reduces or prevents fouling. Transverse outlet 404 is configured such that quench agent flowing into effluent line 114 from transverse outlet 404 is unidirectional with effluent flowing through effluent line 114 from reactor 104. This configuration reduces or to eliminates backmixing of quench agent/effluent into effluent line 114 and quench agent line 118. In at least one embodiment, transverse outlet 404 has a diameter (d₁) that is narrow (e.g., less than 0.25 inches) to ensure high velocity of quench agent as it exits transverse outlet 404 and enters effluent line 114. Quench agent line 118 can have a diameter (d₂) that is narrow (e.g., less than 0.38 inches) to ensure high velocity of quench agent as it flows through quench agent line 118. In at least one embodiment, (d₂) is less than (d₁). In at least one embodiment, a ratio of (d₂) to (d₁) is from 8:1 to 1:1, such as from 2:1 to 1.1. One or more of these configurations reduces or eliminates backmixing of quench agent/effluent into and fouling within effluent line 114 and quench agent line 118. The size of the opening (d₁) can be small enough such that the flow of the quench solution into effluent 114 produces sufficient shear that it reduces or prevents accumulation of solids at the quench/reactor effluent interface.

After sufficient mixing, an effluent is transferred to flash drum 122 via effluent line 124. The amount and flow rate of effluent provided to flash drum 122 is controlled by letdown valve 126. Valve 126 can be any suitable valve, such as a V-ball valve, which can be obtained commercially is the Fisher V-Series from Fisher Valves & Instruments. In some embodiments, the pressure of effluent in effluent line 114 can be 3,000 psi or greater and the temperature can be 175° C. during use. However, a pressure within flash drum 122 can be from 300 psi to 400 psi and a temperature from 100° C. to 150° C. In at least one embodiment, valve 126 is a V-Ball valve which provides a flow path that becomes wider as the valve progresses further into an open position, providing a controllable pressure letdown. This controlled pressure letdown reduces or prevents precipitates from forming in effluent line 114 and/or flash drum 122, which reduces or prevents plugging in effluent line 114 and/or flash drum 122. In at least one embodiment, a V-ball valve is a segmented-ball valve having a V-shaped flow opening such that the width of the flow opening gets larger as the valve opens further. During a linear alpha olefin forming process, a temperature within effluent line 114, mixer 112, quench agent line 118, flash drum 122, and effluent line 124 can be maintained at a temperature of 130° C. or greater to prevent C₃₀₊ waxes and polyethylene from crystallizing out of the process solvent solution. Flash drum 122 will contain process solvent, unreacted ethylene, linear alpha olefins, quenching agent, and any byproducts/impurities (if present) such as polyethylene, branched alpha olefins, linear internal olefins, and C₃₀₊ waxes. At a temperature of 130° C. or greater, ethylene and quenching agent can be volatilized to a top portion of flash drum 122 and can be provided, as an effluent, to knockout drum 125 via effluent line 196.

### Distillation Towers

In addition, distillation towers of the present disclosure (such as distillation tower 194 of FIG. 1B) can comprise one or more dividing walls disposed within the distillation tower(s). The main feedstream(s) to the distillation tower will enter the distillation tower at a location below the top and above the bottom of the dividing wall. The feed will be fractionated in the distillation zone (chamber) formed by that side of the dividing wall. The distillation tower itself, including the separate chambers formed by the dividing wall, can contain any combinations of a plurality of distillation plates, structured corrugated metal packing, or randomly dumped loose packing for separating liquids based on boiling points of the feed into the distillation tower. Above the top of the dividing wall and below the bottom of the dividing wall, vapors and liquids are co-mingled within the distillation tower. Various co-mingled product streams can be removed at varying heights from the distillation tower as desired by the operator. Light streams, including C₄-C₈ hydrocarbons, can be removed at the top of the distillation tower. Heavy streams, including C₁₀-C₂₀ hydrocarbons, can be removed at the bottom of the distillation tower. Middle boiling streams, including a solvent that boils between C₈ and C₁₀ can be removed from an outlet line on the opposite side of the dividing wall from the feed.

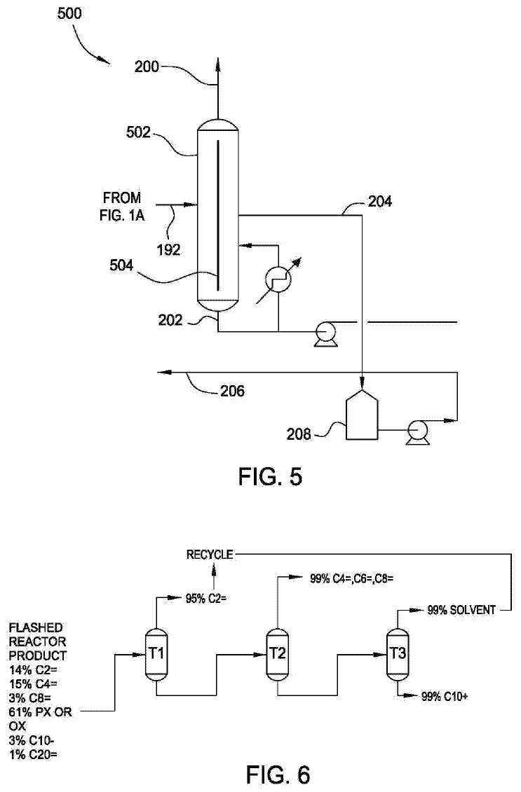

FIG. 5 is a distillation zone of an assembly for producing linear alpha olefins, according to another embodiment of the present disclosure. Distillation zone 500 comprises fractional distillation tower 502 comprising dividing wall 504. Linear alpha olefin products (with remaining process solvent) are transferred, as an effluent, via effluent line 192 (from deethanizer tower 188) to fractional distillation tower 502. During use, fractional distillation tower 502 separates light linear alpha olefins (C₄, C₆, C₈) from heavier linear alpha olefins (C₁₀-C₂₀). The light linear alpha olefins can be removed, as an effluent, from fractional distillation tower 502 via effluent line 200. The light linear alpha olefin fraction can be collected and stored or can undergo further purification in one or more additional distillation tower(s). The heavier linear alpha olefins can be removed, as an effluent, from fractional distillation tower 502 via effluent line 202. The heavier linear alpha olefin fraction can be collected and stored or can undergo further purification in one or more additional distillation tower(s).

Process solvent present in distillation tower 502 can be (as a middle distillate) removed, as an effluent, from fractional distillation tower 502 via effluent line 204. The process solvent can be collected and stored in process solvent storage tank 208 or can be recycled via recycle loop line 206 back to process solvent source 262. In conventional distillation towers, because process solvent, such as paraxylene, boils between C₈ and C₁₀ linear alpha olefins, a middle distillate would contain a minor fraction of C₈ and C₁₀ linear alpha olefins as well as some residual water content. A middle distillate can often have C₈ and C₁₀ olefin content of 4 wt %. However, a fractional distillation tower (such as tower 502) comprising a dividing wall (such as dividing wall 504) provides recovered process solvent in the middle distillate in very high purity, with C₈ and C₁₀ olefin content of less than 0.5 wt %, such as less than 0.05 wt % and water content of less than 10 ppm, such as less than 25 ppb, for recycle to process solvent source 262. In addition to providing surfaces for distillation/condensation to occur, a dividing wall (such as dividing wall 504) also prevents effluent from effluent line 192 directly entering effluent line 204 upon entry of the effluent into distillation tower 194 by blocking the flow of effluent entering the distillation tower on a first side of the distillation tower from flowing directly to a second side of the distillation tower opposite the first side. Because of dividing wall 192, the reduced C₈ and C₁₀ olefin content in the recycled process solvent increases linearity of linear alpha olefins formed in reactor 104. Furthermore, the reduced water content in the recycled process solvent provides reduced hydrolysis of catalysts in reactor 104 and, accordingly, reduced fouling polymer formation. The reduced water content also reduces or eliminates a need for further water reduction (such as by an additional distillation tower) of recycled process solvent before returning the recycled process solvent to process solvent source 262.

### Methods

For methods of the present disclosure, the olefin (ethylene) in a tubular reactor can react (oligomerize) in the presence of the catalyst to form a linear alpha olefin (an oligomer) having two or more monomers bonded together. Depending on the monomer and/or catalyst selected and the reaction conditions maintained in the tubular reactor, the assemblies and methods of the present disclosure may be adapted to oligomerize the monomer into any number of possible oligomers. In one embodiment, the olefin may be ethylene. Ethylene may be oligomerized to form butene (dimerization), hexene (trimerization), octene, decene, and higher-order oligomers. In some embodiments, a catalyst may selectively oligomerize the monomer to a desired oligomer, such as for use as a desired oligomer product. The selectivity of the catalyst may depend on multiple reaction conditions, including the concentration of olefin in a tubular reactor, the residence time of the olefin and oligomers in the tubular reactor, temperature within the tubular reactor, etc. For methods of the present disclosure, any suitable catalyst system and set of reaction conditions may be utilized. Preferably, the oligomerization reaction will be conducted in a manner to maximize the selectivity of a desired linear alpha olefin product.

Ethylene introduced to the tubular reactor should contain less than 1 ppb oxygen and less than 10 ppb water. This level of purity can be achieved using a combination of a suitable copper catalyst and 3 Å molecular sieve before introducing the olefin into the olefin source, such as olefin source 260. Solvent can also contain less than 2 ppb water, which can be achieved by continuously circulating the solvent through beds of molecular sieves, while simultaneously sparging dry nitrogen through the storage vessels. Dry nitrogen can be prepared by circulating the nitrogen over 3 Å molecular sieve until an on-line moisture analyzer indicates a water content below 20 ppbw.

The oligomerization reaction takes place in the tubular reactor with pressure that can be controlled at the reactor outlet to maintain all feed components in a single dense phase. The oligomerization reaction is exothermic with heat being removed through the walls of the tubular reactor. A steam jacket is disposed around the water supply. Heat from the reactor walls vaporizes the fluid (e.g., water) to remove heat from the reaction. This process generates steam. The steam pressure in the steam jacket is controlled to maintain the desired reactor temperature. In one embodiment, the temperature of the reactor is held near 150° C. In one embodiment, the reactor has a minimum temperature during oligomerization of 130° C. (which reduces or prevents polymer crystallization). In one embodiment, the reactor has a maximum temperature in the reactor that is 170° C. in order to maintain a single dense phase. The reactor outlet pressure can be set at 2900 psig, with an inlet pressure of 3000 psig to maintain desired flow rates.

In one embodiment, the solvent to ethylene ratio entering the reactor can be from 0.5 to 1.5, such as 1.0. The total water in the reactor feed is less than 25 ppb by weight. The Al/Zr molar ratio in the reactor feed can be 12. The residence time of the reactants in the reactor can be 10 min. The weight percent of zirconium tetrachloride in the adduct mixture is 0.5% (before introduction into the reactor feed source). The weight percent of zirconium adduct in the process solvent is 2.5%.

The oligomerization reaction can be “quenched” immediately after leaving the reactor. “Quenching” involves rapid deactivation of the active catalyst species at the reactor outlet, before ethylene is flashed off (in a flash drum), which reduces or prevents a loss in product linearity. An organic amine is used as the quench agent. A possible quench agent is 2-methyl-1,5-penta-methylenediamine. The quench agent can be dissolved as a 2 wt % solution with the process solvent and preheated to within 10° C. of the reactor effluent temperature. The quench solution can be fed at a rate that provides a molar ratio of nitrogen in the quench to chlorine in the reactor effluent that is close to 2.0 and not less than 1.0. Injection flow can be as “continuous” as possible (not pulsed), being stabilized via pulsation dampeners and the use of multiple-head injection pumps. Mixing in the letdown valve itself is typically sufficient.

After the reactor quench, the pressure is let down to release unreacted olefin in the vapor phase. The temperature in the flash drum should be high enough to prevent high molecular weight waxes and polyolefin from crystalizing out of solution (>130° C.). The actual flash temperature will vary with olefin conversion in the reactor. For example, in one embodiment, temperature in the flash drum is near 140° C. and the pressure is 25 atm. The overhead of the flash drum can be cooled with refrigerant to from 25° C. to 60° C., for example 35° C., which reduces the amount of quench agent (such as amines) in the recycle gas. The cooled vapor can then be fed to a knock out drum to separate vapor recycle from the condensed material. A demister can be used in the knockout drum headspace to prevent liquid carry-over to the recycle line. The liquid from the knock out drum can be re-heated and fed back into the flash drum through a pump (sparger). A heater can be present on the liquid return line to provide the additional heat to maintain the flash temperature above 130° C.

Liquid from the flash drum can be pressurized to 35 barg and mixed with a recirculating caustic stream by a static mixer and flowed to a settling drum. The temperature in the settling drum can be from 130° C. to 160° C., such as 140° C. The quenched catalyst species are hydrolysed and transferred into the aqueous phase. The organic and aqueous phases are separated in the settling drum. The organic phase can then be washed a second time with the recirculating caustic stream in a second settling drum, to lower the salt content. The caustic solution can be injected in a recycle loop of this second settling drum, and a small spent caustic purge is taken from the first settling drum. The makeup caustic rate should be sufficient to provide 50% excess caustic over stoichiometric amount to solubilize the metals, such as aluminum, in the flash drum effluent. At steady state, the flow of metals in the flash drum effluent is controlled by the catalyst feed rate. The ratio of purge to make-up caustic should be 7:1.

The organic phase of the second settling drum can then be water washed to remove residual salts and quench agent. The temperature of the water wash tower can be from 130° C. to 160° C., such as 140° C. The water wash tower can be configured to remove quench agent, such as amine, to provide an organic phase having 1 ppm or less quench agent. The water wash tower can have from 4 to 8 distillation plates, such as 6 distillation plates. A small water washing effluent stream can be recycled to the second settling drum to compensate for water loss. The main water purge can be transferred to the waste-water facility for further treatment. The organic phase from the washing tower can be transferred to the de-ethanizer tower.

The deethanizer tower can be a conventional distillation column with approximately 15 distillation plates. Refrigeration on the overhead of the column can be used to limit the amount of, for example, butene in the recycle stream. Purge streams can be present in the overhead recycle in order to remove impurities from the olefin feed. A drier on the recycle gas is required to remove any remaining water. This drier system can include two parallel driers such that at any time one drier is active while the other is being regenerated. A source of regeneration gas can be used to heat the molecular sieves to 230° C.

The bottom fraction of the de-ethanizer tower can be fed to a distillation section to recover the process solvent and separate the products. Preferably, the temperature in the distillation section does not go below 130° C. for any streams that contain the high molecular weight polymer byproduct. Preferably, the temperature does not exceed 280° C. to avoid product degradation. The recycle solvent stream should contain less than 1 wt % quench agent in order to avoid poisoning the catalyst. The ratio of recycle solvent to ethylene in the reactor feed should be 1:1. The separation of the reactor products and recovery of the solvent can be achieved through conventional distillation techniques.

Periodically, the reactor can be cleaned with solvent at 195° C. When the reactor is first started up, it can be purged with solvent at 150° C. for 2 days until the exit concentration of water is 25 ppb by weight, such as 15 ppb by weight or less. Before start-up, the reactor can also be treated with catalyst (such as zirconium/aluminum mixture) for 3 days at 15° C.

In methods of the present disclosure, ethylene can be selectively trimerized to form 1-hexene. Other olefins, such as propylene, 1-butene, and 2-butene and the like, may also be trimerized as part of the tubular reactor feed, for example from olefin source 260. Ethylene and/or the other olefins can also be dimerized or tetramerized as part of a reaction according to methods of the present disclosure.

Methods for synthesizing linear alpha olefins of the present disclosure may be performed under generally known oligomerisation conditions of temperature and pressure within a tubular reactor, that is at a temperature from 50° C. to 250° C., for example 170° C., and under a pressure of 3450 kPa to 34,500 kPa (500 to 5,000 psig), preferably from 6900 kPa to 24,100 kPa (1,000 to 3,500 psig).

Methods for synthesizing linear alpha olefins of the present disclosure can be performed in solution in an inert process solvent which should be non-reactive with the catalyst, olefin, and linear alpha olefins, particularly a C₆-C₁₀₀ alpha-olefin. The olefin reactant(s) and/or the catalyst system will generally be fed to the tubular reactor along with the process solvent. For purposes of the present disclosure, a “solvent” includes a material added to the reactor feed, in addition to the catalyst and the olefin. Solvents of the present disclosure typically have a boiling point of from −20° C. to 150° C.

Process solvents can include mineral oil; straight and branched-chain hydrocarbons, such as propane, isobutane, butane, pentane, isopentane, hexane, isohexane, heptane, octane, dodecane, and mixtures thereof; cyclic and alicyclic hydrocarbons, such as cyclohexane, cycloheptane, methylcyclohexane, methylcycloheptane, and mixtures thereof; perhalogenated hydrocarbons such as perfluorinated C₄-C₁₀ alkanes; chlorobenzenes; and aromatic and alkylsubstituted aromatic compounds, such as benzene, toluene, mesitylene, paraxylene, orthoxylene, and metaxylene. Suitable process solvents may additionally or alternately include olefin solvents, which can act as monomers or comonomers in linear alpha olefin formation. Olefin process solvents include ethylene, propylene, 1-butene, 1-hexene, 1-pentene, 3-methyl-1-pentene, 4-methyl-1-pentene, 1-octene, 1-decene, and mixtures thereof. With regard to catalyst solvent and/or diluent, there is flexibility as far as what catalyst solvent and/or diluent may be used.

In one embodiment, where process solvent is present, the process solvent can be advantageously selected from the group consisting of toluene, xylenes, propane, butane, isobutane, pentane, isopentane, hexane, cyclohexane, and combinations thereof. Preferred solvents are toluene, cyclohexane, paraxylene, orthoxylene, and metaxylene. Mixtures of these solvents may also be used.

In at least one embodiment, a process solvent comprises a major fraction of orthoxylene, such as a process solvent comprising an orthoxylene content of 50 vol % or greater, such as 75 vol % or greater, such as 90 vol % or greater, such as 95 vol % or greater, such as 99 vol % or greater, such as 99.9 vol % or greater. It has been discovered that orthoxylene is particularly advantageous because it is non-reactive under linear alpha olefin forming conditions, linear alpha olefins and polymer byproducts are highly soluble in orthoxylene, and orthoxylene is readily separable from linear alpha olefins during distillation. For example, orthoxylene has a boiling point of 291° F., whereas paraxylene has a boiling point of 281° F. During a distillation process, such as in distillation tower 194, 1-octene (boiling point 250° F.) as the top fraction is separated from 1-decene (boiling point 339° F.) as the bottom fraction. Because orthoxylene's boiling point of 291° F. is further away from the boiling point of 1-octene (a difference in temperature of 41° F.) as compared to the boiling point of paraxylene (281° F.) (a difference in temperature of 31° F.), and because the boiling point of orthoxylene is sufficiently far away from that of 1-decene, the middle distillate fraction of orthoxylene has a higher purity than a middle distillate fraction of paraxylene, the middle distillates being obtained under distillation conditions. The relative ease of distilling orthoxylene from 1-octene and 1-decene provides a reduction in energy input into a column for distillation processes (as illustrated in Tables 1 and 2) and provides a process solvent for recycle that has less C⁸ and C¹⁰ linear alpha olefins and water than recycled paraxylene process solvent. FIG. 6 is a distillation scheme using paraxylene or orthoxylene as solvent, according to an embodiment of the present disclosure. Tables 1 and 2 illustrate heat duties (boiler duty and condenser duty) for the columns shown in FIG. 6. As used herein, “heat duty” includes the energy input (units: MM BTU/HR) into a distillation column (the reboiler or the condenser) to generate reflux and distillate. Pro II SimSci™ Simulation software (from Schneider Electric Software, LLC) was used for heat duty calculations. As used herein, “MM BTU/HR” means a “thousand thousand British thermal units per hour”. A BTU is the amount of heat required to increase the temperature of a pint of water (which weighs exactly 16 ounces) by one degree Fahrenheit. As shown in Tables 1 and 2, use of orthoxylene reduces the total reboiler duty by 30% and reduces the total condenser duty by 36%. The lower reboiler and condenser duties with use of orthoxylene as a solvent in methods of the present disclosure provides an energy and cost savings to the assembly owner/operator.

TABLE 1
Paraxylene Solvent

| Column | | | | |
|---|---|---|---|---|
| Name | | T1 | T2 | T3 |
| Condenser | MM BTU/HR | −3.08E−05 | −3.07E−04 | −1.68E−04 |
| Duty | | | | |
| Reboiler | MM BTU/HR | 1.52E−04 | 3.12E−04 | 1.34E−04 |
| Duty | | | | |

TABLE 2
Orthoxylene solvent

| Column | | | | |
|---|---|---|---|---|
| Name | | T1 | T2 | T3 |
| Condenser | MM BTU/HR | −3.08E−05 | −1.34E−04 | −1.60E−04 |
| Duty | | | | |
| Reboiler | MM BTU/HR | 1.45E−04 | 1.49E−04 | 1.26E−04 |
| Duty | | | | |

The olefin, such as ethylene, used in methods of the present disclosure preferably contains not more than the following limits of impurities: acetylenic hydrocarbons less than 1 part per million by weight; dienes less than 1 part per million by weight; carbon monoxide less than 5 parts per million by weight; carbon dioxide less than 15 parts per million by weight; oxygen-containing compounds (e.g. methanol, ethanol, acetone or sec-butanol) less than 1 part per million by weight; water less than 5 parts per million by weight; hydrogen less than 1 part per million by weight; oxygen less than 3 parts per million by weight; sulphur less than 5 milligrams per cubic meter; chlorine less than 5 milligrams per cubic meter.

The water content of the olefin in an olefin source, such as olefin source 260, is to preferably reduced still further to less than 20 parts per billion before it is provided to a tubular reactor, such as reactor 104, e.g., by contacting with 3A or 4A molecular sieves.

The linearity of the linear alpha olefins (the oligomers) formed by methods of the present disclosure can be further improved by introducing into the reactor feed from 10 to 50 parts per million by volume, preferably 20 to 40 parts per million by volume, of oxygen. The oxygen can be introduced into the reactor feed (from a line that is coupled with line 108) before the mixture is introduced into a tubular reactor, such as reactor 104. In such embodiments, the amount of catalyst used can be increased in order to compensate for the reduction of catalyst activity (if any) caused by the oxygen. For example, at 40 ppm of oxygen by volume the catalyst concentration can be doubled to achieve the same degree of conversion as that obtained in the absence of the oxygen. At 20 ppm of oxygen by volume, the proportion of catalyst can be increased by 30%.

The temperature and pressure of the linear alpha olefin formation being performed within a tubular reactor, such as reactor 104, may be varied to adjust the molecular weight and yield of the desired linear alpha olefin. If a two component catalyst system is used, the molecular weight (number average molecular weight (Mn)) of the linear alpha olefins formed in the tubular reactor may be controlled by adjustment of the molar ratio of the second component of the catalyst to the first component (e.g. ratio of aluminum or zinc (co-catalyst) to zirconium (catalyst)).

The preferred reaction temperature for the production of linear alpha olefins of the present disclosure having from 6 to 20 carbon atoms is 120° C. to 250° C. At these temperatures, in a tubular reactor, conversions of 65 to 80% of olefin, such as ethylene, at 120° C. to 250° C. can be achieved at pressures of from 20,000 kPa to 22,000 kPa, such as 20,700 kPa (3,000 psig), depending upon, for example, the particular configuration of the reactor. The amount of catalyst used is conveniently expressed as the weight ratio of the ethylene feed to the metal (such as zirconium) in the catalyst. Generally, from 10,000 to 120,000 parts by weight of olefin, such as ethylene, are used per part by weight of metal (such as zirconium) in the catalyst, the preferred amount being from 25,000 to 35,000 parts by weight of ethylene per part by weight of metal and most preferably 31,000 parts by weight of ethylene.

During the reaction, the mol ratio of the olefin feed to the oligomerisation product should be maintained at 0.8 or greater in order to minimize copolymerization reactions (between olefins and linear alpha olefin products) which might interfere with the achievement of the desired high degree of linearity of the product. Preferably this ratio is greater than 2.

The linear alpha olefin oligomerisation product may be isolated by procedures, e.g., use of an aqueous caustic catalyst quench followed by water washing and recovery of the final product by distillation.

### Catalysts

Catalysts used in methods of the present disclosure can form linear alpha olefins from olefin monomers (such as ethylene) in a tubular reactor. Catalysts of the present disclosure can have an olefin selectivity of at least 95 mol %, for example at least 97 mol % or at least 98 mol % to the desired linear alpha olefin product. Additionally or alternately, the catalyst can have an olefin selectivity of at least 95 mol % to the desired oligomerization product.

Catalysts used in methods of the present disclosure can comprise homogeneous, organometallic systems, such as single site, chromium catalyst systems. Such systems can comprise a chromium source in combination with a heterocyclic, di-aryl, or phosphorus compound such as a pyrrole, pyridyl or pyridyl-phosphino compound, along with an alkyl aluminum activator such as methyl alumoxane (MAO) or modified methyl alumoxane (MMAO). Catalysts can be provided as a pre-formed catalyst system or one or more parts of the catalyst system may be provided to the tubular reactor separately. For example, in some implementations the activator may be provided separately to olefin transfer line 108 via an activator source (not shown) at which point the activator mixes with other reactor feed components (e.g., ethylene and catalyst) flowing through olefin transfer line 108.

Catalysts of methods of the present disclosure may be more or less active upon entering the reactor. The catalyst activity will increase as the chromium source, the alkyl aluminum activator and the olefin mix to form the active catalyst species. This induction period used for the catalyst system to reach its maximum activity can range from 0.5 to 3 hours depending on the reactor conditions. The presence or absence of an induction period, and its relative duration, may affect the optimal residence time of the catalyst in the reactor. In one embodiment, a catalyst is contacted with an activator in catalyst source 266, where an induction period occurs within the catalyst source 266.

Alternatively, in some embodiments, the activator (such as methylalumoxane) can be combined with catalyst immediately prior to introduction into the tubular reactor. Such mixing may be achieved by mixing in line 108, followed by swift injection into the tubular reactor, by mixing in-line just prior to injection into the reactor, or the like. In such embodiments, the catalyst and activator are contacted for less than 0.5 hours before injection into the reactor. Likewise, in situ activation, where the catalyst system components are injected separately into the tubular reactor, with or without olefin, and allowed to combine within the reactor directly, can also be useful. In some embodiments, the catalyst system components are allowed to contact each other for 0.5 hours or less, prior to contact with olefin, alternately for 5 minutes or less, alternately for 3 minutes or less, alternately for 1 minute or less.

As mentioned above, catalysts of the present disclosure can include a two component catalyst in which the first component is an adduct of zirconium, such as ZrClₐBr_{b} wherein each of “a” and “b” is 0, 1, 2, 3 or 4 and a+b=4 with an organic compound of up to 30 carbon atoms selected from the group consisting of esters, ketones, ethers, amines, nitriles, anhydrides, acid chlorides, amides and aldehydes, and a second alkyl metal component selected from the group consisting of R₂AlX, RAlX₂/R₃Al₂X₃, R₃Al, and R₂Zn wherein R is alkyl or 1 to 20 carbon atoms and X is Cl or Br, the oligomerisation being conducted in the presence of 10 to 50 ppm by volume of oxygen based on the ethylene.

The first component of the catalyst can be an adduct of ZrClₐBr_{b}, with an ester, ketone, ether, amine, nitrile, anhydride, acid chloride, amide or aldehyde, and these various adduct-forming organic compounds may have up to 30 carbon atoms. The adduct generally includes mole ratios of organic component to zirconium of from 0.9 to 1 up to 2 to 1. Equimolar adducts are preferred. The adduct must be soluble and stable in the solvent used as the reaction medium for the oligomerisation process of the invention. Suitable zirconium halides include ZrCl₄, ZrBr₄ and mixed halides such as ZrClBr₃, ZrCl₂Br₂ and ZrCl₃Br. Adducts of ZrCl₄ are particularly preferred.

The organic compound used to form the adduct is preferably an ester represented by the formula R¹COOR² wherein R¹ and R² are each alkyl, aryl, alkaryl, aralkyl of 1 to 30 carbon atoms and R¹ may also be hydrogen. R¹ and R² taken together may also represent a cycloaliphatic group and the ester may be a lactone such as gamma-butyrolactone or phthalide. Especially preferred are alkyl acetate esters wherein the alkyl group has 6 to 16 carbon atoms, e.g., n-hexyl acetate, n-heptyl acetate, n-octyl acetate, n-nonyl acetate, n-decyl acetate, isohexyl acetate, isodecyl acetate and the like, which have been found to form dimeric equimolar adducts with ZrCl₄. Particularly preferred adducts may be represented by the formula (ZrCl₄.CH₃COOR¹)₂ where IV is C₆ to C₁₆ alkyl or a mixture thereof. These preferred ester adducts are capable of providing highly concentrated solutions in most process solvents used, e.g., up to 40 percent by weight of ZrCl₄ when the preferred mixed isodecyl acetate esters are used. Particularly useful are mixtures of various isomers of isohexyl, isoheptyl, isooctyl, isononyl, isodecyl or isotridecyl acetate. The adducts may be prepared by simple addition of the organic ester to a mixture of ZrCl₄ and the inert process solvent, such as within catalyst source 266. The ester is added slowly to the stirred mixture at 23° C. and complete formation and dissolution of the adduct is observed within a few minutes. The dissolution is exothermic and the mixture can reach a temperature of 50° C. during the adduct formation.

Also suitable for providing soluble zirconium adducts useful as the first component in a catalyst for methods of the present disclosure are ketones, ethers and aldehydes which may be represented respectively by the formulae R¹COR², R¹OR² and R¹COH wherein IV and R² each represent alkyl, aryl alkaryl or aralkyl and a total number of carbon atoms in R¹ and R² is not more than 30. Also suitable are primary, secondary and tertiary amines wherein the hydrocarbyl radicals have up to 30 carbon atoms such as n-dodecyl amine and tri-n-hexyl amine. Also suitable are hydrocarbyl cycloaliphatic ethers and ketones having from 4 to 16 carbon atoms, e.g., cyclohexanone.

Other adduct forming organic compounds useful for methods of the present disclosure include nitriles and hydrides, acid chlorides and amides having up to 30 carbon atoms. These may be represented by the formulae RCN, (RCO)₂O, RCOCl, RCONH₂, RCONHR and RCONR₂ where R represents a hydrocarbyl, alkyl, aryl, alkaryl or aralkyl group of up to 30 carbon atoms. Examples are n-undecane nitrile, n-decyl succinic anhydride and n-decanoyl chloride.

The second catalyst component useful for methods of the present disclosure is an aluminum alkyl of the formulae R₂AlX, RAlX₂, R₃Al₃X₃, or R₃Al or a zinc alkyl of the formula R₂Zn, where R is an alkyl of 1 to 20 carbon atoms and X is Cl or Br. Diethylaluminum chloride, aluminum ethyl dichloride and mixtures thereof are preferred.

The relative amounts of the two catalyst components used in the process of the invention can be varied. In at least one embodiment, a mol ratio of the second component to the first component from 1:1 up to 50:1, such as from 10:1 to 25:1, where the first component is a zirconium catalyst (or chromium catalyst) and the second component is an aluminum co-catalyst.

The two catalyst component adduct can be formed, for example, in a stirred tank reactor, fitted with a nitrogen sparge and vent, heating and cooling systems and a pump around filter system before being introduced into line 108. The nitrogen sparge system prevents air entry into the unit while charging, under operation and while unloading product. The heating system can be sized to provide the heat in the initial drying step in which the unit is sparged with hot, dry nitrogen. The stirred tank reactor can provide vigorous stirring to keep all the zirconium tetrachloride powder in suspension to ensure complete reaction with solvent (such as ester solvent). The solvent can be dried with molecular sieves before being mixed with the zirconium tetrachloride slurry. The mix ratio can be between 0.860 and 0.903 pounds of ester per pound of zirconium tetrachloride. A cooling system can be disposed on the jacket of this mixing vessel to lower the temperature of the product. The filter system in the pumparound should contain sintered metal support plates and sufficient space to receive a filter aid and zirconium. The aluminum co-catalyst can be in a dry solvent at a concentration of 15 wt % or less. Both the catalyst and co-catalyst can be pumped up to reactor pressures using a diaphragm pump before or while being mixed with process solvent and introduced to line 108 (reactor feed).

Linear alpha olefin formation parameters according to methods of the present disclosure can be selected and controlled, for example by residence time of a mixture within a tubular reactor as well as temperature within the tubular reactor. Methods for producing linear alpha olefins of the present disclosure can yield from 20 mol % to 99 mol %, for example from 40 mol % to 95 mol % or from 60 mol % to 90 mol %, single pass conversion of feed olefin (e.g., ethylene) from a reactor feed, such as from line 108, through a tubular reactor, such as reactor 104. In some embodiments, particularly where a relatively low single pass conversion of feed olefin (e.g., ethylene) is desired, the linear alpha olefin formation parameters can yield a single pass conversion of feed olefin (e.g., ethylene) from 10 mol % to 60 mol %, for example from 10 mol % to 50 mol %, from 10 mol % to 40 mol %, from 20 mol % to 50 mol %, from 20 mol % to 40 mol %, from 30 mol % to 50 mol %, from 25 mol % to 55 mol %, from 35 mol % to 55 mol %, from 35 mol % to 45 mol %, from 25 mol % to 45 mol %, from 20 mol % to 35 mol %, from 10 mol % to 30 mol %, from 15 mol % to 45 mol %, or from 15 mol % to 55 mol %. In other embodiments, particularly where a relatively medium single pass conversion of feed olefin (e.g., ethylene) is desired, the linear alpha olefin formation parameters can yield a single pass conversion of feed olefin (e.g., ethylene) from 30 mol % to 80 mol %, for example from 40 mol % to 70 mol %, from 30 mol % to 60 mol %, from 30 mol % to 50 mol %, from 30 mol % to 70 mol %, from 40 mol % to 60 mol %, from 35 mol % to 75 mol %, from 35 mol % to 65 mol %, from 35 mol % to 55 mol %, from 45 mol % to 75 mol %, from 45 mol % to 65 mol %, from 50 mol % to 80 mol %, from 40 mol % to 75 mol %, or from 50 mol % to 75 mol %.

The desired linear alpha olefins produced via the aforementioned processes may be homopolymerized, used as a comonomer input of a polyolefin (co)polymerization process, and/or utilized in a variety of other applications. In one preferred embodiment, the desired reaction product of linear alpha olefin formation methods of the present disclosure can be C₄-C₂₀ linear alpha olefins, such as 1-butene, 1-hexene, 1-octene, 1-decene, and mixtures thereof. In another preferred embodiment, the desired oligomerization product can comprise 1-hexene, 1-octene, or a combination thereof.

All documents described herein are incorporated by reference herein, including any priority documents and/or testing procedures to the extent they are not inconsistent with this text. As is apparent from the foregoing general description and the specific embodiments, while forms of the present disclosure have been illustrated and described, various modifications can be made without departing from the spirit and scope of the present disclosure. Accordingly, it is not intended that the present disclosure be limited thereby.

1. A method for producing a linear alpha olefin, comprising:
providing an olefin, a catalyst, and a process solvent to a reactor;
obtaining an effluent produced in the reactor; and
transferring the effluent to a solvent-containing portion of a flash drum via a first effluent line coupled with the flash drum.

2. The method of claim 1, further comprising controlling the flow rate of effluent transferred to the flash drum with a V-Ball valve.

3. The method of claim 1, further comprising providing heat to the effluent line using one or more heaters coupled with the effluent line.

4. The method of claim 1, wherein the temperature in the flash drum is 130° C. or greater.

5. The method of claim 4, further comprising transferring an effluent from the flash drum to a knockout drum via an effluent line and cooling the effluent using a chiller coupled with the effluent line.

6. The method of claim 5, further comprising transferring a first effluent from the knockout drum to the reactor and transferring a second effluent from the knockout drum to the flash drum.

7. The method of claim 6, wherein the first effluent from the knockout drum is ethylene.

8. The method of claim 1, further comprising recycling an effluent from the solvent-containing portion of the flash drum by transferring the effluent from the solvent-containing portion of the flash drum via an effluent line to the solvent-containing portion of the flash drum and heating the effluent using a heater coupled with the effluent line.

9. The method of claim 1, further comprising mixing an effluent from the flash drum with a caustic solution.

10. The method of claim 1, further comprising recycling an effluent from the solvent-containing portion of the flash drum by transferring the effluent from the solvent-containing portion of the flash drum via an effluent line to the solvent-containing portion of the flash drum and introducing a quench agent into the effluent line via a quench agent line.

11. The method of claim 1, further comprising obtaining an effluent from the flash drum and transferring the effluent to a caustic solution mixer.

12. The method of claim 11, further comprising obtaining an effluent from the caustic solution mixer and transferring the effluent to a settling drum.

13. The method of claim 12, further comprising obtaining an effluent from the settling drum and transferring the effluent to a distillation tower.

14. The method of claim 13, wherein the distillation tower comprises a dividing wall.

15. The method of claim 14, further comprising providing energy to the distillation tower and obtaining the process solvent from the distillation tower.

16. The method of claim 13, further comprising recycling the process solvent by transferring the process solvent to a process solvent source.

17. The method of claim 1, wherein the process solvent is paraxylene or orthoxylene.

18. The method of claim 1, wherein the catalyst is a chromium catalyst.

19. The method of claim 1, wherein the catalyst is a zirconium catalyst.

20. The method of claim 18, wherein the catalyst further comprises an aluminum catalyst.

21. An assembly for producing linear alpha olefins, the assembly comprising:
a configuration to provide olefin, catalyst and process solvent coupled with a reactor;
a flash drum;
a first effluent line coupled with the reactor at a first end of the first effluent line and coupled with the flash drum at a second end of the first effluent line; and
a second effluent line coupled with the flash drum at a first end of the second effluent line and coupled with the first effluent line at a second end of the second effluent line.

22. The assembly of claim 21, further comprising a heater coupled with the first effluent line.

23. The assembly of claim 21, further comprising a quench agent mixer coupled with the first effluent tube.

24. The assembly of claim 21, further comprising a settling drum coupled with a caustic solution mixer via an effluent line having a first end coupled with the caustic solution mixer and a second end coupled with the settling drum.

25. The assembly of claim 24, further comprising a water tower coupled with the settling drum via an effluent line having a first end coupled with the settling drum and a second end coupled with the water tower.

26. The assembly of claim 25, further comprising a deethanizer coupled with the water tower via an effluent line having a first end coupled with the water tower and a second end coupled with the deethanizer.
